(19) United States
(12) Patent Application Publication
(10) Pub. No.: US 2009/0030416 A1
(43) Pub. Date: Jan. 29, 2009

(54) INTRAMEDULLARY NAIL, METHOD FOR EMBEDDING AND EXTRACTING THE SAME

(76) Inventors: Yingze ZHANG, Shijiangzhuang (CN); Xicheng Li, Shijiangzhuang (CN); Qi Zhang, Shijiangzhuang (CN); Wei Chen, Shijiangzhuang (CN)

Correspondence Address: MERCHANT & GOULD PC, P.O. BOX 2903, MINNEAPOLIS, MN, 55402-0903, US

(21) Appl. No.: 12/122,996

(22) Filed: May 19, 2008

(30) Foreign Application Priority Data
Jul. 27, 2007 (CN) 200710062494.6

(51) Int. Cl. A61B 17/56

(52) U.S. Cl. 606/62

(57) ABSTRACT

Disclosed is an intramedullary nail and a method for embedding and extracting the same through which the problem of having difficulty to correctly determine the position of the intramedullary nail is solved, and the present invention has the following advantages: a correct and rapid determination of the position of the extracting incision, a small incision, a small hurt, a low infection rate, and a quick healing. The intramedullary nail comprises a nail main body; an end cover connected with a tip of the nail main body; a mark body; and a thread connecting the end cover with the mark body. The method for embedding intramedullary nail comprises embedding the thread and the mark body in subcutaneous soft tissues after the nail main body and the end cover are fixed in a tubular bone. And the method for extracting the intramedullary nail comprises determining a position of the mark body, and incising an extracting incision according to the position of the mark body. The position of the mark body is determined by a touch examination, an X-ray examination, or an ultrasonic examination. A surgeon can find the mark body and the thread connected to the mark body after the extracting incision is incised, and incise a path leading to the nail main body along the thread in soft tissues.

### BACKGROUND OF THE INVENTION

1. Field of the Invention

The present invention mainly relates to an orthopaedics medical appliance, particularly relates to an intramedullary nail used in a long tubular bone reposition internal fixation treatment and a method for embedding and extracting the same.

2. Description of the Related Art

At present, an intramedullary nail is a preferred internal fixation nail used in a long tubular bone (such as a thighbone) fracture treatment. Fractured ends of a tubular bone can evenly bear an axial pressure by elastically fixing the intramedullary nail in an axis of the bone, so that bending resistance and rotating resistance are improved, harmful shearing stress and harmful twisting stress are avoided, and a stress-sheltering effect resulting from eccentric fixation is overcome as much as possible. Further, an incision exposure time is short when the intramedullary nail is embedded by a closed-reduction or small-incision reduction operation. Thus infection of the incision during the clinical procedure may be reduced, and a very high fracture-healing rate is obtained.

Therefore, the intramedullary nail has solved many complicated and difficult problems of internal fixation of long tubular bones, and has been widely used in long bone fracture treatments.

Basic structure shape of various intramedullary nails is a long column body which may be straight, or may be slightly curved so as to accommodate to a tubular bone of a certain curvature. An end cover is provided on a tip of the column body. The end cover is exposed outside of the tubular bone after the intramedullary nail is embedded. A hole in the end cover is used to be in screwed connection with a nail extractor when extracting the intramedullary nail.

The end cover is embedded in muscles, and usually is wrapped by peripheral scar tissues or callus, which results in a great trouble of determining a position of the end cover when the nail is extracted. Particularly, when the muscles near the end cover (or the tip of the intramedullary nail) are thick, it is more difficult to determine the position of the intramedullary nail by touch. Accordingly, during extracting the intramedullary nail, an incision for extracting the intramedullary nail is large, an amount of bleeding is excessive, and there is also a potential risk of damaging peripheral nerves and blood vessels.

The more extensive is a clinical use of the intramedullary nail, the more obvious is the problem of having difficulty to correctly determining the position of the intramedullary nail when extracting the intramedullary nail. Thus, there is a need for solving the above problem.

### SUMMARY OF THE INVENTION

In view of the above, at least an object of the present invention is to provide an intramedullary nail a position of which can be correctly determined, and a method for embedding and extracting the same, so that the problem of having difficulty to correctly determining the position of the intramedullary nail can be solved.

According to an aspect of the present invention, there is provided an intramedullary nail comprising: a nail main body; an end cover connected with a tip of the nail main body; a mark body; and a thread connecting the end cover with the mark body.

Preferably, a position of the mark body may be determined by a touch examination, an X-ray examination, or an ultrasonic examination.

Preferably, the mark body has an annular shape or a sheet shape and is made of a soft medical material.

Preferably, the mark body has a conspicuous color.

Preferably, the thread is a soft medical thread with high strength.

Further preferably, the medical thread with high strength is a ceramic thread with high strength, or a macromolecule synthesis thread with high strength.

Preferably, the thread has a conspicuous color.

According to another aspect of the present invention, there is provided a method for embedding an intramedullary nail. The intramedullary nail comprises a nail main body; an end cover connected with a tip of the nail main body; a mark body; and a thread connecting the end cover with the mark body. The method comprises: embedding the thread and the mark body in subcutaneous soft tissues after the nail main body and the end cover are fixed into a tubular bone.

According to still another aspect of the present invention, there is provided a method for extracting an intramedullary nail. The intramedullary nail comprises a nail main body; an end cover connected with a tip of the nail main body; a mark body; and a thread connecting the end cover with the mark body. The method comprises: determining a position of the mark body, and incising an extracting incision according to the position of the mark body.

Preferably, a position of the mark body can be determined by a touch examination, an X-ray examination, or an ultrasonic examination.

Further preferably, the extracting method further comprises: finding the mark body and the thread connected to the mark body after the extracting incision is incised, incising a path in soft tissues along the thread, the path leading to the end cover and the nail main body, and pulling out the nail main body and the end cover via the path by means of the thread.

The present invention also relates to a medical appliance. The medical appliance comprises a main body, which needs to be extracted after being embedded in a human body or an animal body; a mark body; and a thread, which connects the mark body with the main body.

In the present invention, the thread and the mark body are added to the tip of the intramedullary nail, so that the position of an incision for extracting the intramedullary nail can be correctly determined by means of simple examination(s); and the intramedullary nail can be pulled out by a path incised along the thread. The intramedullary nail according the present invention has all the advantages of the current intramedullary nail. At the same time, the thread and the mark body can be applied to various intramedullary nails because the thread and the mark body are available and the application thereof to the intramedullary nail is convenient.

To apply the intramedullary nail and the embedding and extracting methods for the same according the present invention to treat a tubular bone fracture can produce the following advantages.

1. The position of the extracting incision can be very correctly and rapidly determined, and thus an incision length of an extracting incision can be reduced as much as possible, and a long incision, a large hurt, an excessive bleeding amount, damages to periphery nerves and blood vessels, and other disadvantages affecting curative effect which result from inaccurately locating the intramedullary nail can be effectively prevented.

2. A postoperative infection rate can be remarkably reduced because the extracting incision is small and the hurt is small, and a healing speed of the incision is remarkably improved.

3. An operation cost can be remarkably reduced and an operation time is shortened, because the intramedullary nail can be extracted in a state of local anesthesia for the reason of the small incision.

4. The thread and the mark body may both have conspicuous colors, and thus the thread and the mark body can be seen by a surgeon upon incising muscles, so that the surgeon can incise a path leading to the intramedullary nail along the thread rapidly and accurately.

5. The thread and the mark body are made of soft medical materials, and thus there are no bad feelings and no rejection reaction with the muscles after the embedding, and the thread and the mark body can not be absorbed. Therefore, the thread and the mark body are safe in utilization, and do not affect functional trainings after operation.

### BRIEF DESCRIPTION OF THE DRAWINGS

FIG. 1 is a schematic view showing a structure of an intramedullary nail according to an embodiment of the present invention.

### REFERENCE NUMERALS IN THE FIGURE ARE:

1 nail main body

2 end cover

3 thread

4 mark body

### DETAILED DESCRIPTION OF THE EMBODIMENTS

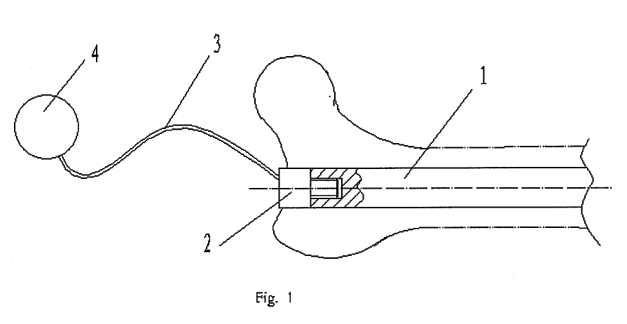

FIG. 1 is a schematic view showing a structure of an intramedullary nail according to an embodiment of the present invention.

As shown in FIG. 1, the intramedullary nail according to the present invention comprises a nail main body 1, an end cover 2 connected with a tip of the nail main body 1, a thread 3 and a mark body 4, wherein the thread 3 connects the end cover 2 with the mark body 4. The intramedullary nail can have various structure forms, such as an interlocking intramedullary nail, an intramedullary nail with an inner core, and the like. The basic structure shape of the nail main body is a substantially elongated column body with an end cover on the tip.

The thread 3 can be a soft medical-use thread with high strength, such as a medical ceramic thread with high strength, or a medical-use macromolecule synthesis thread with high strength.

Here, the mark body 4 has an annular shape or a sheet shape, or other shapes without sharp edges. The mark body 4 is made of a soft medical material. After being embedded, a position of the mark body 4 can be determined by a touch examination, an X-ray examination, an ultrasonic examination, or other simple auxiliary examinations.

The thread 3 and the mark body 4 may not cause bad feelings and there is no rejection reaction with the muscles after the embedding, and the thread can not be absorbed. Therefore, the thread is safe in utilization, and does not affect functional trainings after operation.

Now the connection manner of the thread 3 and the end cover 2 will be explained. The thread 3 and the end cover 2 can be formed integrally directly during manufacture or can be fixed with the end cover 2 by a mechanical structure on the end cover 2.

The mechanical structure can be the following:

a hole is provided between an end face of the end cover 2 and a side face of the end cover, a diameter of the hole is slightly larger than that of the thread 3 so that the thread 3 can be connected with the end cover 2 through the hole;

a cross beam is formed on the end face of the end cover 2 by providing a groove in the end face, then the thread 3 can be tied onto the cross beam through the groove, or a cross beam structure is provided directly on the end face of the end cover 2 through which the thread 3 can pass;

an axial through hole is provided in the end cover 2 substantially along an axis of the end cover 2, a diameter of the axial through hole is slightly larger than that of the thread 3 and the thread has one enlarged end of a diameter larger than that of the axial through hole so that the thread 3 can be attached to the end cover 3 by passing through the axial through hole so that the enlarged end is held by the hole; or

a screw thread fit structure, i.e., a screw and an internal thread in the end cover 2 fitting with the screw, the thread 3 is fixed to the end cover 2 by winding it on the screw and then screwing the screw into the internal thread.

It should be noted that the way to connecting the thread 3 and the end cover 2 is not limited to the above, and any other ways suitable to connect the thread 3 and the end cover 2 obvious to those skilled in the art can also be used.

Now the connection manner of the thread 3 and the mark body 4 will be explained. The thread 3 and the mark body 4 can be formed integrally directly during manufacture or can be directly tied to the annular mark body 4, or can be fixed with the mark body 4 by a mechanical structure on the end cover 2, such as a hole, an annular structure, a rod shaped structure.

Of course, the way for connecting the thread 3 and the mark body 4 is not limited to the above, and any other ways suitable to connect the thread 3 and the mark body 4 obvious to those skilled in the art can also be used.

Now a method for embedding the intramedullary nail will be explained.

Generally, the method for embedding the intramedullary nail comprises the following steps: incising an embedding incision in an appropriate position; embedding the intramedullary nail body 1 into a tubular bone and fixing the nail main body 1; fixing the end cover 2 onto the nail main body 1; and to stitch the embedding incision.

As for the intramedullary nail of the present invention, however, the method for embedding the intramedullary nail further comprises the following step: embedding the thread 3 and the mark body 4 in subcutaneous soft tissues after the nail main body 1 and the end cover 2 are fixed into the tubular bone. Preferably, the embedding the thread 3 and the mark body 4 in the subcutaneous soft tissues comprises placing the thread 3 and the mark body 4 in an axial direction of the nail main body 1 so that the thread 3 and the mark body 4 extend toward outside of the skin.

Now a method for extracting the intramedullary nail will be explained.

Generally, the method for extracting the intramedullary nail comprises the following steps: incising an extracting incision, detaching a fastener of the intramedullary nail, pulling out the nail body 1 and the end cover 2, and stitching the extracting incision.

As for the intramedullary nail of the present invention, however, the step of incising an extracting incision comprises the following: determining a position of the mark body 4, and incising an extracting incision according to the position of the mark body 4. Preferably, the method for extracting the intramedullary nail further comprises the following steps: finding the mark body 4 and the thread 3 connected to the mark body 4 after the extracting incision is incised, incising a path leading to the end cover 2 and the nail main body 1 along the thread 3 in soft tissues, and pulling out the nail main body 1 and the end cover 2 via the path by means of the thread 3. Wherein, the position of the mark body 4 can be determined by a touch examination, an X-ray examination, or an ultrasonic examination.

It should be noted that the mark body 4 and the thread 3 may both have conspicuous colors so as to facilitate finding the mark body 4 and incising a path in soft tissues along the thread 3 during extracting the intramedullary nail.

In the process of extracting the intramedullary nail, if the nail main body 4 has become loose with time, and there is no callus formation formed around the end cover 2, the nail main body 1 can be pulled out directly by the thread 3; if there is callus formation formed around the end cover 2, the callus formation should be removed first so that the end cover 2 is exposed, then the nail main body 1 can be pulled out by the thread 3; if the intramedullary nail does not become loose so that the nail body can not be pulled out directly, other end of the thread 3 can be wound on a bobbin-like handle, then the intramedullary nail can be pulled out through the handle; and if it is difficult to pull out the nail main body through the handle, a threaded end of a specific puller is connected with the nail main body 1 after the end cover 2 is detached, then the nail main body 1 can be pulled out by the puller.

Unlike the prior art (to determine the position of the extracting incision blindly), according to the present invention, the exact position of the mark body 4 is determined according to a fracture healing X-ray film or the ultrasonic examination, or by the touch examination, and thus the incision of the extracting incision is directed by the correctly determined position of the mark body 4. After the extracting incision is incised, a soft tissue path leading to the nail main body 1 can be incised according to the positions of the mark body 4 and the thread with minimal damage, and the nail main body 1 can be easily found. Therefore, the present invention has overcome the disadvantages in prior art such as a large incision, an excessive bleeding amount, and a potential risk of damaging peripheral nerves and blood vessels.

It should be noted that the present invention is not limited to the intramedullary nail, for example, the present invention may relate to a medical appliance The medical appliance comprises a main body, which needs to be extracted after being embedded in a human body or an animal body; a mark body; and a thread, which connects the mark body with the main body. The mark body and the thread can have features as described above.

Although a few embodiments have been shown and described, it would be appreciated by those skilled in the art that changes and variants may be made in these embodiments without departing from the principles and spirit of the invention, the scope of which is defined in the claimed and their equivalents.

1. An intramedullary nail, comprising:
a nail main body;
an end cover connected with a tip of the nail main body;
a mark body; and
a thread connecting the end cover with the mark body.

2. The intramedullary nail as claimed in claim 1, wherein a position of the mark body can be determined by a touch examination, an X-ray examination, or an ultrasonic examination.

3. The intramedullary nail as claimed in claim 2, wherein the mark body has an annular shape or a sheet shape and is made of a soft medical material.

4. The intramedullary nail as claimed in claim 2, wherein the mark body has a conspicuous color.

5. The intramedullary nail as claimed in claim 1, wherein the thread is a soft medical thread with high strength.

6. The intramedullary nail as claimed in claim 5, wherein the medical thread with high strength is a ceramic thread with high strength, or a macromolecule synthesis thread with high strength.

7. The intramedullary nail as claimed in claim 6, wherein the thread has a conspicuous color.

8. A method for embedding an intramedullary nail, the intramedullary nail comprising a nail main body; an end cover connected with a tip of the nail main body; a mark body; and a thread connecting the end cover with the mark body, wherein the method comprising:
embedding the thread and the mark body in subcutaneous soft tissues after the nail main body and the end cover are fixed into a tubular bone.

9. The method as claimed in claim 8, wherein the embedding the thread and the mark body in the subcutaneous soft tissues comprises placing the thread and the mark body in an axial direction of the nail main body so that the thread and the mark body extend toward outside of a skin.

10. The method as claimed in claim 8, wherein a position of the mark body can be determined by a touch examination, an X-ray examination, or an ultrasonic examination.

11. The method as claimed in claim 10, wherein the mark body has an annular shape or a sheet shape and is made of a soft medical material.

12. The method as claimed in claim 10, wherein the mark body has a conspicuous color.

13. The method as claimed in claim 8, wherein the thread is a soft medical thread with high strength.

14. The method as claimed in claim 13, wherein the medical thread with high strength is a ceramic thread with high strength, or a macromolecule synthesis thread with high strength.

15. The method as claimed in 14, wherein the thread has a conspicuous color.

16. A method for extracting an intramedullary nail, the intramedullary nail comprising a nail main body; an end cover connected with a tip of the nail main body; a mark body; and a thread connecting the end cover with the mark body, wherein the method comprising:
determining a position of the mark body, and incising an extracting incision according to the position of the mark body.

17. The method as claimed in claim 16, wherein the position of the mark body can be determined by a touch examination, an X-ray examination, or an ultrasonic examination.

18. The method as claimed in claim 17, further comprising:
finding the mark body and the thread connected to the mark body after the extracting incision is incised, incising a path along the thread in soft tissues, the path leading to the end cover and the nail main body, and pulling out the nail main body and the end cover via the path by means of the thread.

19. The method as claimed in claim 18, wherein the mark body has an annular shape or a sheet shape and is made of a soft medical material.

20. The method as claimed in claim 18, wherein the mark body has a conspicuous color.

21. The method as claimed in claim 16, wherein the thread is a soft medical thread with high strength.

22. The method as claimed in claim 21, wherein the medical thread with high strength is a ceramic thread with high strength, or a macromolecule synthesis thread with high strength.

23. The method as claimed in 22, wherein the thread has a conspicuous color.

24. A medical appliance, comprising
a main body, which needs to be extracted after being embedded in a human body or an animal body;
a mark body; and
a thread, which connects the mark body with the main body.

25. The medical appliance as claimed in claim 24, wherein a position of the mark body can be determined by a touch examination, an X-ray examination, or an ultrasonic examination.

26. The medical appliance as claimed in claim 25, wherein the mark body has an annular shape or a sheet shape and is made of a soft medical material.

27. The medical appliance as claimed in claim 25, wherein the mark body has a conspicuous color.

28. The medical appliance as claimed in any one of claims 24-27, wherein the thread is a soft medical thread with high strength.

29. The medical appliance as claimed in claim 28, wherein the medical thread with high strength is a ceramic thread with high strength, or a macromolecule synthesis thread with high strength.

30. The medical appliance as claimed in claim 29, wherein the thread has a conspicuous color.
